(19) United States
(12) Patent Application Publication
(10) Pub. No.: US 2022/0023208 A1
(43) Pub. Date: Jan. 27, 2022

(54) PHARMACEUTICAL COMPOSITION FOR TOPICAL USE COMPRISING AT LEAST ONE ANTIINFLAMMATORY SUBSTANCE

(71) Applicant: SOCIETE D'EXPLOITATION DE PRODUITS POUR LES INDUSTRIES CHIMIQUES SEPPIC, Paris Cedex 07 (FR)

(72) Inventors: Catherine BULCOURT, Paris (FR); Alice DENIS, Paris (FR); Séverine SIGURANI, Paris (FR)

(21) Appl. No.: 17/276,693

(22) PCT Filed: Sep. 12, 2019
(86) PCT No.: PCT/FR2019/052116

§ 371 Date: Mar. 16, 2021

(30) Foreign Application Priority Data
Sep. 17, 2018 (FR) 1858341

(51) Int. Cl. A61K 9/107; A61K 9/00; A61K 31/196; A61K 31/407; A61K 31/381; A61K 31/192; A61K 31/23

(52) U.S. Cl. CPC A61K 9/107; A61K 9/0014; A61K 31/196; A61K 31/23; A61K 31/381; A61K 31/192; A61K 31/407

(57) ABSTRACT

A pharmaceutical composition for topical use which is in the form of a water-in-oil emulsion including, for 100% of the weight thereof: —from 60% to 80% by weight of a gelled aqueous phase including an anti-inflammatory substance, —from 20% to 40% by weight of a fatty phase including at least one oil and an emulsifying system including a combination of at least one emulsifying surfactant selected from the elements of the group consisting of alkylpolyglycoside compositions and alkylpolyglycoside and fatty alcohol compositions, and of at least one emulsifying surfactant selected from the elements of the group consisting of polyglycerol esters, alkoxylated polyglycerol esters, polyglycol polyhydroxystearates, polyglycerol polyhydroxystearates and alkoxylated polyglycerol polyhydroxystearates.

### CROSS-REFERENCE TO RELATED APPLICATIONS

This application is the U.S. national phase of International Application No. PCT/FR2019/052116 filed Sep. 12, 2019 which designated the U.S. and claims priority to FR 1858341 filed Sep. 17, 2018, the entire contents of each of which are hereby incorporated by reference.

### BACKGROUND OF THE INVENTION

### Field of the Invention

The present invention relates to a pharmaceutical composition for topical use which is in the form of an emulsion of water-in-oil type comprising at least one anti-inflammatory substance and is intended to be used for therapy in a human being or animal.

### Description of the Related Art

Dermopharmaceutical and pharmaceutical compositions can be in the form of aqueous solutions, emulsions and powders. Emulsions are preferred forms since they make it possible to convey both the water-soluble substances and liposoluble substances that are frequently used in these applications. A distinction is made between oil-in-water (O/W) emulsions in which the continuous phase consists of a hydrophilic phase, generally an aqueous phase, and the dispersed phase consists of a lipophilic fatty phase, and water-in-oil (W/O) emulsions in which the continuous phase consists of a lipophilic fatty phase and the dispersed phase consists of a hydrophilic phase, generally an aqueous phase.

Oil-in-water emulsions are intrinsically more stable than water-in-oil emulsions; water-in-oil emulsions nevertheless have a number of advantages. Specifically, the separation between the water droplets reduces the possibility of the proliferation of microorganisms. In addition, the use of preservatives, which is essential when the continuous phase is aqueous, can be avoided or reduced when the continuous phase is fatty. Water-in-oil emulsions are far less sensitive to low temperature than oil-in-water emulsions. Lastly, for topical applications for cosmetic use, European patent application published under the number EP 1961455 A1 discloses that an oily continuous phase makes it possible to cover the skin after application of the water-in-oil emulsion, which protects the skin from dehydration and against external substances by forming a persistent oily film, thus making it possible to treat dry skin.

The solutions proposed in the prior art for preparing dermatological emulsions which are in the water-in-oil form are not satisfactory because either the silicone derivatives employed are volatile and may have harmful effects with regard to the environment and the users, or the silicone derivatives employed are less volatile and then impart unpleasant sensorial properties after topical application, such as for example sticky sensations on the skin. In addition, when they comprise particular therapeutic substances, such as for example particular nonsteroidal anti-inflammatory substances (or NSAIDs) and more particularly arylacetic (or arylalkanoic) derivatives and 2-arylpropionic acids (or profens), these emulsions of water-in-oil type exhibit stability problems during storage or even an inability to achieve a stable water-in-oil form.

Proceeding from this, one problem is to develop a new composition which is in the form of an emulsion of water-in-oil type, which does not feature the drawbacks exposed above, which remains homogeneous at ambient temperature (25° C.) and at 45° C. after storage for a minimum of three months, and which comprises, as active therapeutic agent, substances chosen from elements of the group consisting of specific nonsteroidal anti-inflammatory agents (or NSAIDs) and more particularly of arylacetic (or arylalkanoic) derivatives and 2-arylpropionic acids (or profens).

### SUMMARY OF THE INVENTION

A solution of the present invention is a pharmaceutical composition for topical use which is in the form of an emulsion of water-in-oil type comprising:
from 60% to 80% by weight of a gelled aqueous phase (A1) comprising a nonsteroidal anti-inflammatory substance (or NSAID) and more particularly by arylacetic (or arylalkanoic) derivatives and 2-arylpropionic acids (or profens),
from 20% to 40% by weight of a fatty phase (A2) comprising at least one oil and an emulsifying system comprising a combination of at least one emulsifying surfactant (S₁) selected from the elements of the group consisting of compositions of alkyl polyglycosides, and compositions of alkyl polyglycosides and of fatty alcohols, and of at least one emulsifying surfactant (S₂) selected from the elements of the group consisting of polyglycerol esters, alkoxylated polyglycerol esters, polyglycol polyhydroxystearates, polyglycerol polyhydroxystearates, and alkoxylated polyglycerol polyhydroxystearates.

### DESCRIPTION OF THE PREFERRED EMBODIMENTS

Preferably, said composition for topical use preferably comprises from 65% to 80% by weight, and more preferentially still from 68% by weight to 80% by weight, of the aqueous phase (A1), and preferably from 20% to 35% by weight, and more preferentially still from 20% to 32% by weight, of the fatty phase (A2).

Depending on the case, the composition for topical use according to the invention may have one or more of the following features:
the gelled aqueous phase comprises, per 100% of its weight, from 0.625% to 10% by weight of a crosslinked anionic polyelectrolyte, from 0.625% to 5% by weight of the anti-inflammatory substance, and from 85% to 98.75% by weight of water; it is noted that the gelled aqueous phase comprises preferably from 0.625% to 8.5% by weight, and more preferentially still from 0.625% to 7% by weight, of a crosslinked anionic polyelectrolyte (PA), preferably from 0.625% to 3.5% by weight, and more preferentially still from 0.625% to 2%, of the inflammatory substance, and preferably from 88% to 98.75% by weight, and more preferentially still from 91% to 98.75% by weight, of water;
the crosslinked anionic polyelectrolyte comprises a proportion of greater than or equal to 25 mol % of monomer units derived from 2-methyl-2-[(1-oxo-2-propenyl)amino]-1-propanesulfonic acid in free acid or partially or totally salified form;
the emulsifying system comprises, per 100% by weight, from 12% to 88% by weight of surfactant (S1) and from 12% to 88% by weight of surfactant (S2);
the fatty phase (A2) comprises from 1.25% to 25% by weight, more particularly from 1.25% to 20% by weight, of the emulsifying system and from 75% to 98.75% by weight, more particularly from 80% to 98.75% by weight, and more particularly still from 82% to 98.75% by weight, of at least one oil and optionally of at least one wax;
the anti-inflammatory substance is chosen from the alkali metal, alkaline earth metal, ammonium, N,N-dialkylammonium and N,N,N-trialkylammonium salts, the alkyl groups of these each comprising between 1 and 4 carbon atoms, of the elements of the group consisting of 2-[2-(2,6-dichlorophenyl)aminophenyl]ethanoic acid (CAS number=15307-86-5) or diclofenac of formula (Ia):

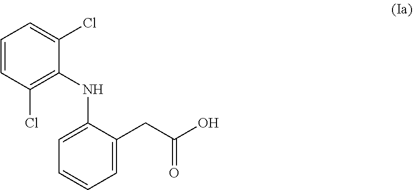 [SMILES: O=C(O)Cc1ccccc1Nc1c(Cl)cccc1Cl]

2-[2-[2-(2,6-dichloroanilino)phenyl]acetyl]oxyacetic acid (CAS number=89796-99-6) or aceclofenac of formula (Ib),

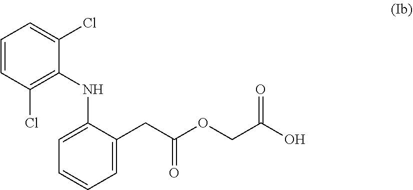 [SMILES: O=C(O)COC(=O)Cc1ccccc1Nc1c(Cl)cccc1Cl]

2-(5-benzoylthiophen-2-yl)propanoic acid (CAS number=33005-95-7 (RS)) or tiaprofenic acid in the R enantiomer form of formula (Ic1) and in the S enantiomer form of formula (Ic2)

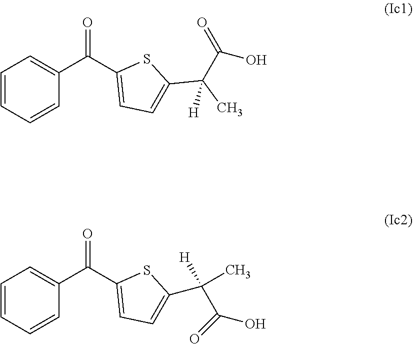 [SMILES: C[C@H](C(=O)O)c1ccc(C(=O)c2ccccc2)s1 ; C[C@@H](C(=O)O)c1ccc(C(=O)c2ccccc2)s1]

2-[4-(2-methylprop-2-enylamino)phenyl]propanoic acid (CAS number=39718-89-3) or alminoprofen of formula (Id),

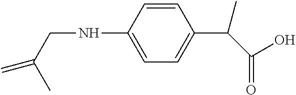 [SMILES: C=C(C)CNc1ccc(C(C)C(=O)O)cc1]

2-(1,8-diethyl-4,9-dihydro-3H-pyrano[3,4-b]indol-1-yl)acetic acid (CAS number=41340-25-4) or etodolac of formula (Ie),

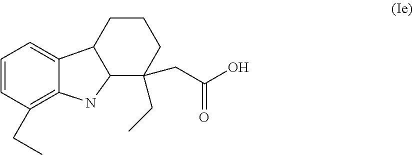 [SMILES: CCc1cccc2c1NC1C2CCCC1(CC)CC(=O)O]

(±)-2-fluoro-α-methyl-(1,1′-biphenyl)-4-acetic acid or flurbiprofen in the R enantiomer form of formula (If1) and in the S enantiomer form of formula (If2), (CAS number=5104-49-4 (RS)):

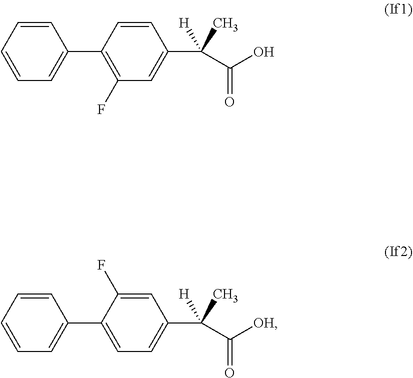 [SMILES: C[C@@H](C(=O)O)c1ccc(-c2ccccc2)c(F)c1 ; C[C@@H](C(=O)O)c1ccc(-c2ccccc2)c(F)c1]

2-[4-(2-methylpropyl)phenyl]propanoic acid or ibuprofen in the R enantiomer form of formula (Ig1) and in the S enantiomer form of formula (Ig2), (CAS number=15687-27-1 (RS)):

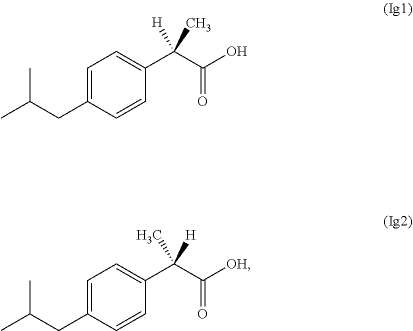

2-(3-benzoylphenyl)propionic acid in the S(+) enantiomer form (CAS number=22161-81-5) and R(−) enantiomer form (CAS number=56105-81-8) and in the form of a racemic mixture (CAS number=22071-15-4) or ketoprofen of formula (Ih),

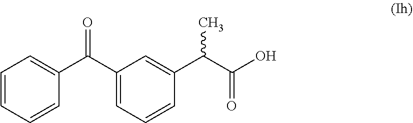 [SMILES: CC(C(=O)O)c1cccc(C(=O)c2ccccc2)c1]

6-methoxy-α-methyl-2-naphthaleneacetic acid or naproxen in the S(+) enantiomer form (CAS number=22204-53-1) of formula (Ii) or in the form of a racemic mixture (CAS number=23981-80-8):

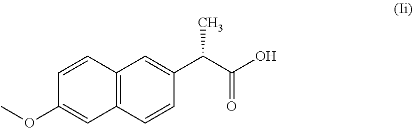 [SMILES: COc1ccc2cc([C@H](C)C(=O)O)ccc2c1]

the anti-inflammatory substance is chosen from the sodium salt of 2-[2-(2,6-dichlorophenyl)aminophenyl]ethanoic acid, the diethylammonium salt of 2-[2-(2,6-dichlorophenyl)aminophenyl]ethanoic acid, the sodium salt of 2-[2-[2-(2,6-dichloroanilino)phenyl]acetyl]oxyacetic acid, the diethylammonium salt of 2-[2-[2-(2,6-dichloroanilino)phenyl]acetyl]oxyacetic acid, the sodium salt of 2-[4-(2-methylpropyl)phenyl]propanoic acid, and the sodium salt of 2-(3-benzoylphenyl)propionic acid; the anti-inflammatory substance is preferably the sodium salt of 2-[2-(2,6-dichlorophenyl)aminophenyl]ethanoic acid.
the emulsifying surfactant (S1) consists of at least one alkyl polyglycoside composition (C1) represented by formula (VIII):

R1-O-(G)x-H (VIII)

in which x, the degree of polymerization, represents a decimal number between 1.05 and 2.5, G represents the glucosyl or β-D-glucopyranosyl radical, obtained from the removal of the hemiacetal hydroxyl group from β-D-glucopyranose, and R1 represents a radical chosen from the elements of the group consisting of the radicals n-dodecyl, n-tetradecyl, n-hexadecyl, n-octadecyl, n-eicosyl and n-behenyl, said composition (C1) consisting of a mixture of compounds represented by the formulae (VIII1), (VIII2), (VIII3), (VIII4) and (VIII5):

R1-O-(G)1-H (VIII1)

R1-O-(G)2-H (VIII2)

R1-O-(G)3-H (VIII3)

R1-O-(G)4-H (VIII4)

R1-O-(G)5-H (VIII5)

in the respective molar proportions a1, a2, a3, a4 and a5, such that:
the sum a1+a2+a3+a4+a5 is equal to 1, and that
the sum a1+2a2+3a3+4a4+5a5 is equal to x.
the emulsifying surfactant (S1) consists of at least one composition (C2) comprising, per 100% of its weight:
   from 10% to 50% by weight of at least one alkyl polyglycoside composition (C1) represented by formula (VIII):

R1-O-(G)x-H (VIII)

in which x represents a decimal number between 1.05 and 2.5, G represents a glucose residue, and R1 represents a radical chosen from the elements of the group consisting of the radicals n-dodecyl, n-tetradecyl, n-hexadecyl, n-octadecyl, n-eicosyl and n-behenyl, said composition consisting of a mixture of compounds represented by the formulae (VIII1), (VIII2), (VIII3), (VIII4) and (VIII5):

R1-O-(G)1-H (VIII1)

R1-O-(G)2-H (VIII2)

R1-O-(G)3-H (VIII3)

R1-O-(G)4-H (VIII4)

R1-O-(G)5-H (VIII5)

in the respective molar proportions a1, a2, a3, a4 and a5, such that:
the sum a1+a2+a3+a4+a5 is equal to 1, and that
the sum a1+2a2+3a3+4a4+5a5 is equal to x; and
from 90% to 50% by weight of at least one fatty alcohol of formula (IX):

R′1-OH (IX),

in which R′1 represents a radical chosen from the elements of the group consisting of the radicals n-dodecyl, n-tetradecyl, n-hexadecyl, n-octadecyl, n-eicosyl and n-behenyl, where R′1 may be identical to or different from R1.
the emulsifying surfactant (S₂) consists of at least one polyglycol polyhydroxystearate represented by the formula (XII):

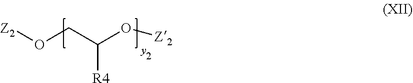

in which y₂ represents an integer greater than or equal to 2 and less than or equal to 50, R₄ represents a hydrogen atom, a methyl radical or an ethyl radical, and Z₂ represents a radical of formula (XIII):

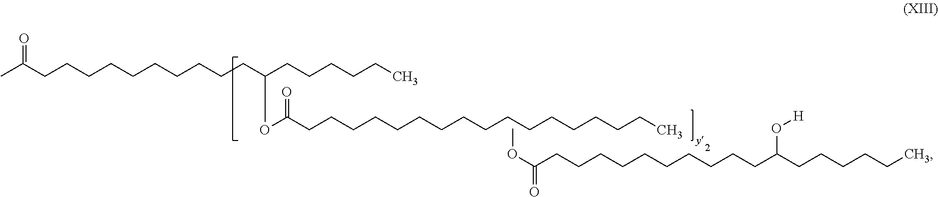

in which y′₂ represents an integer greater than or equal to 0 and less than or equal to 10, more particularly greater than or equal to 1 and less than or equal to 10, and Z′₂ represents a radical of formula (XIII) as defined above, where Z₂′ may be identical to or different from Z₂, or a hydrogen atom.
the weight ratio between the emulsifying surfactant (S1) and the emulsifying surfactant (S2) is greater than or equal to ¼ and less than or equal to 1, preferably greater than or equal to ⅓ and less than or equal to 1, more preferentially still greater than or equal to ⅓ and less than or equal to ½;
said composition is intended to be used for therapy in a human being or animal.
said composition is used for reducing and/or eliminating local pains, post-traumatic inflammation of the joints, muscles, tendons or ligaments, localized forms of soft-tissue rheumatism, localized forms of osteoarthritis, actinic keratosis caused by overexposure to sunlight, acute migraine, pain associated with bone metastases, fever due to malignant lymphogranulomatosis (Hodgkin's lymphoma), multi-drug resistant E. coli, Shy-Drager syndrome and diabetes mellitus.
said composition additionally comprises at least one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizing agents, opacifying agents, pearlescent agents, superfatting agents, sequestering agents, chelating agents, antioxidants, fragrances, essential oils, preservatives, conditioning agents, deodorants, mineral fillers or pigments. The composition according to the invention can generally comprise excipients and/or active principles commonly used in the field of formulations for topical, in particular pharmaceutical or dermopharmaceutical, use.

Among the anti-inflammatory substances which can be used in the context of the invention, mention may be made more particularly of the sodium, potassium, calcium, magnesium, ammonium, N,N-dimethylammonium and N,N-diethylammonium salts of the elements of the group consisting of 2-[2-(2,6-dichlorophenyl)aminophenyl]ethanoic acid (CAS number=15307-86-5) or diclofenac of formula (Ia), 2-[2-[2-(2,6-dichloroanilino)phenyl]acetyl]oxyacetic acid (CAS number=89796-99-6) or aceclofenac of formula (Ib), 2-(5-benzoylthiophen-2-yl)propanoic acid (CAS number=33005-95-7 (RS)) or tiaprofenic acid in the R enantiomer form of formula (Ic₁) and in the S enantiomer form of formula (Ic₂), 2-[4-(2-methylprop-2-enylamino)phenyl]propanoic acid (CAS number=39718-89-3) or alminoprofen of formula (Id), 2-(1,8-diethyl-4,9-dihydro-3H-pyrano[3,4-b]indol-1-yl)acetic acid (CAS number=41340-25-4) or etodolac of formula (Ie), (±)-2-fluoro-α-methyl-(1,1′-biphenyl)-4-acetic acid or flurbiprofen in the R enantiomer form of formula (If₁) and in the S enantiomer form of formula (If₂), (CAS number=5104-49-4 (RS)), 2-[4-(2-methylpropyl)phenyl]propanoic acid or ibuprofen in the R enantiomer form of formula (Ig₁) and in the S enantiomer form of formula (Ig₂), (CAS number=15687-27-1 (RS)), 2-(3-benzoylphenyl)propionic acid in the S(+) enantiomer form (CAS number=22161-81-5) and R(−) enantiomer form (CAS number=56105-81-8) and in the form of a racemic mixture (CAS number=22071-15-4) or ketoprofen of formula (Ih), 6-methoxy-α-methyl-2-naphthaleneacetic acid or naproxen in the S(+) enantiomer form (CAS number=22204-53-1) of formula (Ii) or in the form of a racemic mixture (CAS number=23981-80-8).

In the definition of the composition (E₁) for topical use which is a subject of the present invention, “oil” denotes a compound and/or a mixture of compounds insoluble in water and liquid at 25° C., and more particularly:
linear alkanes comprising from 11 to 19 carbon atoms;
branched alkanes comprising from 7 to 40 carbon atoms, such as isododecane, isopentadecane, isohexadecane, isoheptadecane, isooctadecane, isononadecane or isoeicosane, or mixtures of some of them such as those mentioned below and identified by their INCI name: C₇₋₈ isoparaffin, C₈₋₉ isoparaffin, C₉₋₁₁ isoparaffin, C₉₋₁₂ isoparaffin, C₉₋₁₃ isoparaffin, C₉₋₁₄ isoparaffin, C₉₋₁₆ isoparaffin, C₁₀₋₁₁ isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁₂₋₂₀ isoparaffin, C₁₃₋₁₄ isoparaffin, C₁₃₋₁₆ isoparaffin;
cycloalkanes optionally substituted with one or more linear or branched alkyl radicals;
white mineral oils, such as those sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes comprising from 15 to 19 carbon atoms, said alkanes being linear alkanes, branched alkanes and cycloalkanes, and more particularly the mixture (M₁) which comprises, per 100% of its weight, a proportion by weight of branched alkanes of greater than or equal to 90% and less than or equal to 100%; a proportion by weight of linear alkanes of greater than or equal to 0% and less than or equal to 9%, and more particularly less than 5%, and a proportion by weight of cycloalkanes of greater than or equal to 0% and less than or equal to 1%, for example the mixtures sold under the name Emogreen™ L15 or Emogreen™ L119;
the fatty alcohol ethers of formula (II):

Z₁—O—Z₂ (II),

in which Z₁ and Z₂, which may be identical or different, represent a linear or branched alkyl radical comprising from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
monoesters of fatty acids and of alcohols of formula (III):

R′₁—(C═O)—O—R′₂ (III),

in which R′₁—(C═O) represents a saturated or unsaturated, linear or branched acyl radical comprising from 8 to 24 carbon atoms, and R′₂ represents, independently of R′₁, a saturated or unsaturated, linear or branched hydrocarbon-based chain comprising from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
diesters of fatty acids and of glycerol of formula (IV) and of formula (V):

R′₃—(C═O)—O—CH₂—CH(OH)—CH₂—O—(C═O)—R′₄ (IV)

R′₅—(C═O)—O—CH₂—CH[O—(C═O)—R′₆]—CH₂—OH (V),

in which formulae (VI) (VII) R′₃—(C═O), R′₄—(C═O), R′₅—(C═O) and R′₆—(C═O), which may be identical or different, represent a saturated or unsaturated, linear or branched acyl group comprising from 8 to 24 carbon atoms;
triesters of fatty acids and of glycerol of formula (VI):

R′₇—(C═O)—O—CH₂—CH[O—(C═O)—R″₈]—CH₂—O—(C═O)—R″₉ (VI),

in which R′₇—(C═O), R′₈—(C═O) and R′₉—(C═O), which may be identical or different, represent a saturated or unsaturated, linear or branched acyl group comprising from 8 to 24 carbon atoms.
vegetable oils, such as phytosqualane, sweet almond oil, coconut kernel oil, castor oil, jojoba oil, olive oil, rapeseed oil, peanut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, oils derived from flowers or vegetables;
ethoxylated vegetable oils;

When the composition according to the invention comprises a wax, the latter is more particularly selected from beeswax, carnauba wax, candelilla wax, ouricoury wax, Japan wax, cork fiber wax, sugar cane wax, paraffin waxes, lignite waxes, microcrystalline waxes, lanolin wax; ozokerite; polyethylene wax; silicone waxes; vegetable waxes; fatty alcohols and fatty acids that are solid at ambient temperature; glycerides that are solid at ambient temperature.

Preferably, the composition according to the invention comprises at least one oil chosen from the elements of the group consisting of castor oil, liquid paraffins, cocoyl caprate/caprylate, isopropyl myristate and capric/caprylic triglyceride.

In the definition of the composition according to the invention, the term “crosslinked anionic polyelectrolyte (PA)” denotes a non-linear crosslinked anionic polyelectrolyte, which is in the form of a three-dimensional network which is insoluble in water but swellable in water and which leads to the production of a chemical gel.

Preferably, the crosslinked anionic polyelectrolyte comprises, per 100 mol %:

(a1)—a proportion of greater than or equal to 25 mol % and less than or equal to 100 mol % of monomer units derived from 2-methyl-2-[(1-oxo-2-propenyl)amino]-1-propanesulfonic acid in free acid or partially or totally salified form;
(a2)—optionally, a proportion of greater than 0 mol % and less than or equal to 75 mol % of monomer units derived from at least one monomer chosen from the elements of the group consisting of acrylamide, N,N-dimethylacrylamide; methacrylamide and N-isopropylacrylamide;
(a3)—optionally, a proportion of greater than 0 mol % and less than or equal to 20 mol %, more particularly greater than 0 mol % and less than or equal to 15 mol %, more particularly still greater than or equal to 0 mol % and less than or equal to 10 mol % of monomer units derived from at least one monomer chosen from the elements of the group consisting of (2-hydroxyethyl) acrylate, (2,3-dihydroxypropyl) acrylate, (2-hydroxyethyl) methacrylate, (2,3-dihydroxypropyl) methacrylate and vinylpyrrolidone;
(a4)—optionally, a proportion of greater than 0 mol % and less than or equal to 75 mol % of monomer units derived from at least one monomer chosen from the elements of the group consisting of acrylic acid, methacrylic acid, 2-carboxyethylacrylic acid, itaconic acid, maleic acid and 3-methyl-3-[(1-oxo-2-propenyl)amino]butanoic acid, the carboxylic function of said monomers being in free acid, partially salified or totally salified form;
(a5) optionally, a proportion of greater than 0 mol % and less than or equal to 5 mol % of at least one monomer of formula (VII):

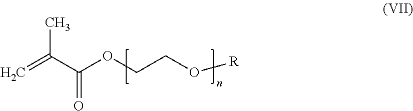

in which R represents a linear or branched alkyl radical comprising from 8 to 20 carbon atoms and n represents an integer greater than or equal to zero and less than or equal to 20;
(a6)—a proportion of greater than 0 mol % and less than or equal to 1 mol % of monomer units derived from at least one diethylenic or polyethylenic crosslinking monomer (AR); the sum of said molar proportions of the monomer units a1), a2), a3), a4), a5) and a6) being equal to 100 mol %.

For the purposes of the present invention, the term “salified” indicates that the acid function present in a monomer is in an anionic form associated in salt form with a cation, in particular alkali metal salts, such as sodium or potassium cations, or such as nitrogenous base cations, such as the ammonium salt, the lysine salt or the monoethanolamine salt (HOCH₂—CH₂—NH₄). They are preferably sodium or ammonium salts.

The term “at least one diethylenic or polyethylenic crosslinking monomer (AR)” notably denotes, in the definition of said crosslinked anionic polyelectrolyte (P), a monomer chosen from the elements of the group consisting of methylenebis(acrylamide), ethylene glycol dimethacrylate, diethylene glycol diacrylate, ethylene glycol diacrylate, diallylurea, triallylamine, trimethylolpropane triacrylate, diallyloxyacetic acid or a salt thereof, such as sodium diallyloxyacetate, or a mixture of these compounds; and more particularly a monomer chosen from ethylene glycol dimethacrylate, triallylamine, trimethylolpropane triacrylate or methylenebis(acrylamide) or a mixture of these compounds.

According to another particular aspect of the present invention, composition (E₁) for topical use is characterized in that said crosslinking monomer (AR) as defined previously is used in a molar proportion of less than or equal to 0.5%, more particularly less than or equal to 0.25% and most particularly less than or equal to 0.1%; it is more particularly greater than or equal to 0.005 mol %.

The crosslinked anionic polyelectrolyte (PA) used in the composition (E₁) for topical use which is a subject of the present invention may also comprise various additives such as complexing agents, transfer agents or chain-limiting agents.

The crosslinked anionic polyelectrolyte (PA) used in the composition (E₁) for topical use which is a subject of the present invention may be prepared by performing a radical polymerization process known to those skilled in the art, for instance processes of solution polymerization, suspension polymerization, inverse suspension polymerization, emulsion polymerization, inverse emulsion polymerization or polymerization in solvent medium followed by a step of precipitation of the polymer formed.

According to a more particular aspect, the crosslinked anionic polyelectrolyte (PA) used in the composition (E₁) for topical use which is a subject of the present invention may be prepared by performing a process of polymerization in solvent medium followed by a step of precipitation of the polymer formed, or of inverse emulsion polymerization optionally followed by a step of concentration and/or atomization.

According to a more particular aspect, the crosslinked anionic polyelectrolyte (PA) used in the composition (E₁) for topical use which is a subject of the present invention may be prepared according to one of the processes described above and may involve the use of transfer agents or chain-limiting agents. The transfer agents or chain-limiting agents are more particularly chosen from the group consisting of sodium hypophosphite, alcohols of low molecular weight, for example methanol, ethanol, 1-propanol, isopropanol or butanol, thiols, for example 2-mercaptoethanol, transfer agents comprising a sulfate function, for example sodium methallylsulfonate, or mixtures of said transfer agents. The transfer agents or chain-limiting agents are more particularly used in molar proportions, expressed relative to the total number of moles of monomers used, of 0.001 mol % to 1 mol %, more particularly of 0.001 mol % to 0.5 mol %, and most particularly from 0.001 mol % to 0.1 mol %.

According to another particular aspect of the present invention, said crosslinked anionic polyelectrolyte (PA) is an element of the group consisting of a homopolymer of 2-methyl-2-[(1-oxo-2-propenyl)amino]-1-propanesulfonic acid partially or totally salified in sodium salt or ammonium salt form, crosslinked with triallylamine and/or methylenebis(acrylamide); a copolymer of 2-methyl-2-[(1-oxo-2-propenyl)amino]-1-propanesulfonic acid partially or totally salified in sodium salt or ammonium salt form and of acrylic acid partially or totally salified in sodium salt or ammonium salt form, crosslinked with triallylamine and/or methylenebis(acrylamide); a copolymer of 2-methyl-2-[(1-oxo-2-propenyl)amino]-1-propanesulfonic acid (γ) partially or totally salified in sodium salt or ammonium salt form and of acrylic acid (δ) partially or totally salified in sodium salt form in a molar ratio (γ)/(δ) of greater than or equal to 30/70 and less than or equal to 90/10, crosslinked with triallylamine and/or methylenebis(acrylamide); a copolymer of 2-methyl-2-[(1-oxo-2-propenyl)amino]-1-propanesulfonic acid (γ) partially or totally salified in sodium salt form, and of acrylic acid (δ) partially or totally salified in sodium salt form in a molar ratio (γ)/(δ) of greater than or equal to 40/60 and less than or equal to 90/10, crosslinked with triallylamine and/or methylenebis(acrylamide); a copolymer of 2-methyl-2-[(1-oxo-2-propenyl)amino]-1-propanesulfonic acid (γ) partially or totally salified in sodium salt form, and of acrylamide (ε) in a molar ratio (γ)/(ε) of greater than or equal to 30/70 and less than or equal to 90/10, crosslinked with triallylamine and/or methylenebis(acrylamide); a copolymer of 2-methyl-2-[(1-oxo-2-propenyl)amino]-1-propanesulfonic acid (γ) partially or totally salified in sodium salt form, and of hydroxyethyl acrylate (ζ) in a molar ratio (γ)/(ζ) of greater than or equal to 30/70 and less than or equal to 90/10, crosslinked with triallylamine and/or methylenebis(acrylamide); a terpolymer crosslinked with triallylamine and/or methylenebis(acrylamide), of 2-methyl-2-[(1-oxo-2-propenyl)amino]-1-propanesulfonic acid partially or totally salified in sodium salt or ammonium salt form, of acrylamide and of acrylic acid partially or totally salified in sodium salt or ammonium salt form; a terpolymer crosslinked with triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 30% and less than or equal to 45%, of acrylamide in a molar proportion of greater than or equal to 45% and less than or equal to 68% and of acrylic acid partially or totally salified in sodium salt or ammonium salt form in a molar proportion of greater than or equal to 2% and less than or equal to 10%; a terpolymer crosslinked with triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 30% and less than or equal to 45%, of acrylamide in a molar proportion of greater than or equal to 47% and less than or equal to 68% and of acrylic acid partially or totally salified in sodium salt or ammonium salt form in a molar proportion of greater than or equal to 2% and less than or equal to 8%; a terpolymer crosslinked with trimethylolpropane triacrylate and/or triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 60% and less than or equal to 80%, of N,N-dimethylacrylamide in a molar proportion of greater than or equal to 15% and less than or equal to 39.5% and of tetraethoxylated lauryl methacrylate in a molar proportion of greater than or equal to 0.5% and less than or equal to 5%; a tetrapolymer crosslinked with trimethylolpropane triacrylate and/or triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 60% and less than or equal to 80%, of N,N-dimethylacrylamide in a molar proportion of greater than or equal to 15% and less than or equal to 39%, of lauroyl methacrylate in a molar proportion of greater than or equal to 0.5% and less than or equal to 2.5%, and of stearoyl methacrylate in a molar proportion of greater than or equal to 0.5% and less than or equal to 2.5%.

Regarding the emulsifying surfactant (S1), in formula (VIII) as defined above, the group R1-O— is linked to G via the anomeric carbon of the saccharide residue, so as to form an acetal function.

According to one particular aspect, in the definition of formula (VIII) x, or mean degree of polymerization, represents a decimal number greater than or equal to 1.05 and less than or equal to 2.5, more particularly greater than or equal to 1.05 and less than or equal to 2.0 and more particularly still greater than or equal to 1.25 and less than or equal to 2.0.

According to another particular aspect, the combination of at least one emulsifying surfactant (S1) and at least one emulsifying surfactant (S2) comprises, per 100% of its weight:
from 12% to 88% by weight of at least one emulsifying surfactant (S1) which consists of at least one composition (C₂) comprising, per 100% of its weight:
from 10% to 50% by weight of at least one alkyl polyglycoside composition (C₁) represented by formula (VIII):

R₁—O-(G)x-H (VIII)

in which x represents a decimal number between 1.05 and 2.5, G represents a glucose residue, and R₁ represents a radical chosen from the elements of the group consisting of the radicals n-dodecyl, n-tetradecyl, n-hexadecyl, n-octadecyl, n-eicosyl and n-behenyl, said composition consisting of a mixture of compounds represented by the formulae (VIII₁), (VIII₂), (VIII₃), (VIII₄) and (VIII₅):

R₁—O-(G)₁-H (VIII₁)

R₁—O-(G)₂-H (VIII₂)

R₁—O-(G)₃-H (VIII)

R₁—O-(G)₄-H (VIII₄)

R₁—O-(G)₅-H (VIII₅)

in the respective molar proportions a₁, a₂, a₃, a₄ and a₅, such that:
the sum a₁+a₂+a₃+a₄+a₅ is equal to 1, and that
the sum a₁+2a₂+3a₃+4a₄+5a₅ is equal to x; and
   from 90% to 50% by weight of at least one fatty alcohol of formula (IX):

R′₁—OH (IX),

in which R′₁ represents a radical chosen from the elements of the group consisting of the radicals n-dodecyl, n-tetradecyl, n-hexadecyl, n-octadecyl, n-eicosyl and n-behenyl, where R′₁ may be identical to or different from R₁;
from 12% to 88% by weight of at least one emulsifying surfactant (S₂) which consists of at least one polyglycol polyhydroxystearate represented by the formula (XII):

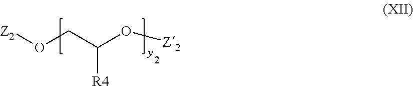

in which y₂ represents an integer greater than or equal to 2 and less than or equal to 50, R₄ represents a hydrogen atom, a methyl radical or an ethyl radical, and Z₂ represents a radical of formula (XIII):

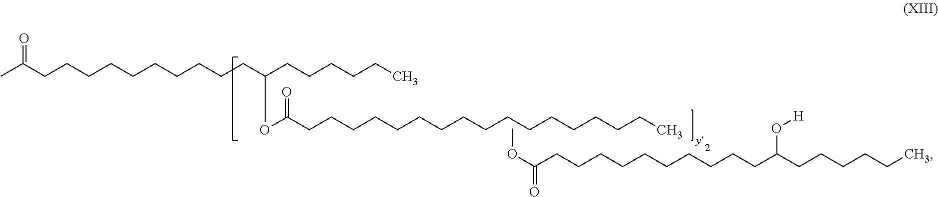

in which y′₂ represents an integer greater than or equal to 0 and less than or equal to 10, more particularly greater than or equal to 1 and less than or equal to 10, and Z′₂ represents a radical of formula (XIII) as defined above, where Z₂′ may be identical to or different from Z₂, or a hydrogen atom.
According to another particular aspect, the composition according to the invention has a dynamic viscosity, measured at a temperature of 20° C. using a Brookfield LVT viscometer at a speed of 6 rpm, greater than or equal to 500 mPa·s and less than or equal to 40 000 mPa·s.

A subject of the invention is also a device which is in a form chosen from a pot, a pump-bottle, a wipe, a mask, a transdermal device, a patch, a poultice, a compress, a tube, spray, said device comprising a pharmaceutical composition according to the invention.

The composition according to the invention is intended for topical use and may be incorporated in any type of dermopharmaceutical or pharmaceutical formulation intended for topical use or else in any type of support intended to be placed in contact with the skin (paper, wipe, textile, transdermal device, etc.).

The expression “for topical use” means that the composition (E₁) is used by application to the skin, scalp or mucous membranes, whether this is a direct application in the case of a dermopharmaceutical or pharmaceutical formulation or an indirect application, for example in the case of a product which is in the form of a textile article, for example a wipe, a compress, a patch or a poultice, or a paper article, for example a paper for sanitary use.

The composition according to the invention can be packaged in pressurized form in an aerosol device or in a device of “pump-bottle” type, in a tube, in a device equipped with a perforated wall, for example a grill, or in a device equipped with a ball applicator (known as a “roll-on”).

The composition according to the invention may additionally comprise excipients and/or active principles commonly used in the field of formulations for topical use, in particular pharmaceutical or dermopharmaceutical formulations.

Regarding the auxiliary compounds, among the foaming and/or detergent anionic surfactants that may be combined with the composition according to the invention, mention may be made of alkali metal salts, alkaline-earth metal salts, ammonium salts, amine salts or amino alcohol salts of alkyl ether sulfates, of alkyl sulfates, of alkylamido ether sulfates, of alkylaryl polyether sulfates, of monoglyceride sulfates, of alpha-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkyl sulfosuccinates, of alkyl ether sulfosuccinates, of alkylamide sulfosuccinates, of alkyl sulfoacetates, of alkyl sarcosinates, of acyl isethionates, of N-acyl taurates, of acyl lactylates, of N-acylated derivatives of amino acids, of N-acylated derivatives of peptides, of N-acylated derivatives of proteins, or of fatty acids.

Among the foaming and/or detergent amphoteric surfactants optionally present in the composition according to the invention, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants optionally present in the composition according to the invention, mention may be made particularly of quaternary ammonium derivatives.

Among the foaming and/or detergent nonionic surfactants optionally present in the composition according to the invention, mention may be made more particularly of alkyl polyglycosides containing a linear or branched, saturated or unsaturated aliphatic radical and comprising from 8 to 12 carbon atoms; castor oil derivatives, polysorbates, coconut kernel amides and N-alkylamines.

As examples of thickening and/or gelling surfactants optionally present in the composition according to the invention, mention may be made of:
optionally alkoxylated fatty esters of alkyl polyglycosides, and most particularly ethoxylated esters of methyl polyglucoside such as PEG 120 methyl glucose trioleate and PEG 120 methyl glucose dioleate sold, respectively, under the names Glucamate™ LT and Glumate™ DOE120;
alkoxylated fatty esters such as the PEG 150 pentaerythrityl tetrastearate sold under the name Crothix™ DS53, or PEG 55 propylene glycol oleate sold under the name Antil™ 141;
fatty-chain polyalkylene glycol carbamates such as PPG 14 laureth isophoryl dicarbamate sold under the name Elfacos™ T211, or PPG 14 palmeth 60 hexyl dicarbamate sold under the name Elfacos™ GT2125.

As examples of emulsifying surfactants optionally present in the composition according to the invention, mention may be made of nonionic surfactants, anionic surfactants and cationic surfactants.

As examples of emulsifying nonionic surfactants optionally present in the composition according to the invention, ethoxylated castor oil and ethoxylated hydrogenated castor oil, for example the product sold under the name Simulsol™ 989; compositions comprising glycerol stearate and stearic acid poly(ethoxylated) with between 5 mol and 150 mol of ethylene oxide, for example the composition comprising stearic acid (ethoxylated) with 135 mol of ethylene oxide and glycerol stearate sold under the name Simulsol™ 165; ethoxylated sorbitan esters, for example the products sold under the name Montanox™; ethoxylated mannitan esters; sucrose esters; methyl glucoside esters.

As examples of emulsifying anionic surfactants optionally present in the composition according to the invention, mention may be made of decyl phosphate, cetyl phosphate sold under the name Amphisol™, glyceryl stearate citrate; cetearyl sulfate; the arachidyl/behenyl phosphates and arachidyl/behenyl alcohols composition sold under the name Sensanov™ WR; soaps, for example sodium stearate or triethanolammonium stearate, or N-acylated derivatives of amino acids which are salified, such as for example stearoyl glutamate.

As examples of emulsifying cationic surfactants optionally present in the composition according to the invention, mention may be made of amine oxides, quaternium-82, cetyltrimethylammonium chloride, hexadecyltrimethylammonium bromide, cetylpyridinium chloride, benzalkonium chloride, benzethonium chloride, and the surfactants described in the document WO96/00719 and mainly those in which the fatty chain comprises at least 16 carbon atoms.

As examples of opacifying agents and/or pearlescent agents optionally present in the composition according to the invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols comprising from 12 to 22 carbon atoms.

As examples of texturing agents optionally present in the composition according to the invention, mention may be made of N-acylated derivatives of amino acids, for example lauroyl lysine sold under the name Aminohope™ LL, octenyl starch succinate sold under the name Dryflo™, myristyl polyglucoside sold under the name Montanov 14, cellulose fibers, cotton fibers, chitosan fibers, talc, sericite and mica.

As examples of solvents and co-solvents optionally present in the composition according to the invention, mention may be made of water, organic solvents, for example glycerol, diglycerol, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, mixtures of water and of said organic solvents, propylene carbonate, ethyl acetate, benzyl alcohol and dimethyl sulfoxide (DMSO).

As examples of agents for improving the skin penetration optionally present in the composition according to the invention, mention may be made of glycol ethers, for instance ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono-n-butyl ether, diethylene glycol monoethyl ether (or Transcutol-P), fatty acids such as oleic acid, fatty acid esters of glycerol, for instance glyceryl behenate, glyceryl palmitostearate, behenoyl macroglycerides, polyoxyethylene-2-stearyl ether, polyoxyethylene-2-oleyl ether, terpenes, for instance D-limonene, and essential oils, for instance essential oil of eucalyptus.

As examples of thickeners and/or gelling agents optionally present in the composition according to the invention, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

As examples of thickeners and/or gelling agents optionally present in the composition according to the invention, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, glucosaminoglycans.

As examples of thickening and/or gelling agents optionally present in the composition according to the invention, mention may be made of cellulose, cellulose derivatives such as methyl cellulose, ethyl cellulose, hydroxypropyl cellulose, silicates, starch, hydrophilic starch derivatives, and polyurethanes.

As examples of stabilizers optionally present in the composition according to the invention, mention may be made of microcrystalline waxes, and more particularly of ozokerite, and mineral salts such as sodium chloride or magnesium chloride.

As examples of thermal or mineral waters which can be combined with the composition according to the invention, mention may be made of thermal or mineral waters having a mineralization of at least 300 mg/I, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

As examples of active agents which can be combined with the composition according to the invention, mention may be made of substances or compositions which provide a beneficial effect to the human or animal subject. These active agents may for example be antibodies, analgesics, anti-inflammatories, cytokines, cytoxins, growth factors, hormones, lipids, oligonucleotides, polymers, polysaccharides, polypeptides, protease inhibitors, vitamins, insect repellents, antibiotics or antifungal agents.

As examples of analgesic and anti-inflammatory agents which can be combined with the composition according to the invention, mention may be made of acetaminophen, aspirin, salicylic acid, methyl salicylate, choline salicylate, glycol salicylate, 1-menthol, camphor, mefenamic acid, fluphenamic acid, indomethacin, protizidic acid, fentiazac, tolmetin, tiaprofenic acid, phenylbutazone, oxyphenbutazone, clofezone, pentazocin, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone and betamethasone.

As examples of antiseptic agents which can be combined with the composition according to the invention, mention may be made of cetrimide, povidone-iodine, chlorhexidine, iodine, benzalkonium chloride, benzoic acid, nitrofurazone, benzoyl peroxide, hydrogen peroxide, hexachlorophene, phenol, resorcinol and cetylpyridinium chloride.

As examples of insecticidal agents which can be combined with the composition according to the invention, mention may be made of trichlorfon, triflumuron, fenthion, bendiocarb, cyromazine, diflubenzuron, dicyclanil, fluazuron, amitraz, deltamethrin, cypermethrin, chlorfenvinphos, flumethrin, ivermectin, abamectin, avermectin, doramectin, moxidectin, zeta-cypermethrin, diazinon, spinosad, imidacloprid, nitenpyram, pyriproxyfen, fipronil, cythioate, lufenuron, selamectin, milbemycin oxime, chlorpyrifos, coumaphos, propetamphos, alpha-cypermethrin, cypermethrin high cis, ivermectin, diflubenzuron, cyclodiene, carbamate and benzoylurea.

As examples of antimicrobial agents which can be combined with the composition according to the invention, mention may be made of sulfonamides, aminoglycosides such as for example neomycin, tobramycin, gentamicin, amikacin, kanamycin, spectinomycin, paromomycin, netilmicin, polypeptides, cephalosporins, oxazolidinones such as for example ciprofloxacin, levofloxacin, and ofloxacin.

As examples of active agents which can be combined with the composition according to the invention, mention may be made of vitamin E, coenzyme Q10, L-carnitine, choline, folic acid, magnesium and its salts, caprylic acid, linoleic acid, lauric acid, taurine, vitamin C, vitamin A and vitamins of group B.

The composition according to the invention and as defined above is obtained by performing the preparation process comprising the following steps:

A step a) of preparing the fatty phase (A₂) by mixing all of the elements constituting it in the desired proportions. This mixing step is generally conducted at a temperature of greater than or equal to 20° C. and less than or equal to 80° C., more particularly greater than or equal to 25° C. and less than or equal to 80° C., and more particularly still greater than or equal to 30° C. and less than or equal to 80° C.; it is performed with mechanical stirring at a moderate speed of greater than or equal to 50 rpm and less than or equal to 100 rpm;

A step b) of preparing the aqueous phase (A₁) from all of the elements constituting it in the desired proportions. This mixing step is generally conducted at a temperature of greater than or equal to 20° C. and less than or equal to 80° C., more particularly greater than or equal to 20° C. and less than or equal to 60° C., and more particularly still greater than or equal to 20° C. and less than or equal to 40° C.; it is performed with mechanical stirring at a moderate speed of greater than or equal to 500 rpm and less than or equal to 3000 rpm. In particular, the aqueous phase (A₁) obtained on conclusion of step b) has a dynamic viscosity, measured at 20° C. using a Brookfield LV viscometer at a speed of 6 rpm, of greater than or equal to 200 mPa·s and less than or equal to 40 000 mPa·s, more particularly greater than or equal to 1000 mPa·s and less than or equal to 40 000 mPa·s, and more particularly still greater than or equal to 2000 mPa·s and less than or equal to 40 000 mPa·s;
A step c) during which the fatty phase (A₂) is added to the aqueous phase (A₁) at a temperature of greater than or equal to 20° C. and less than or equal to 80° C., more particularly greater than or equal to 20° C. and less than or equal to 60° C., and more particularly still greater than or equal to 20° C. and less than or equal to 40° C., with mechanical stirring at a moderate speed of greater than or equal to 50 rpm and less than or equal to 400 rpm, so as to obtain the composition according to the invention.
   The examples that follow illustrate the invention without, however, limiting it.

### Preparation and Evaluation of Water-in-Oil Emulsions According to the Invention and of Comparative Water-in-Oil Emulsions.

### 1) Preparation of the Water-in-Oil Emulsions

Three water-in-oil emulsions according to the invention, denoted (F₁) to (F₃), and seven water-in-oil emulsions according to the prior art, denoted (F′₁) to (F′₇), the proportions by weight of the constituents of which are recorded in table 1 below, the contents by weight of the polyelectrolytes being indicated as a percentage of polymeric solids, are prepared by performing the following process.

The constituents of the fatty phase are introduced successively into a beaker, mixed and brought to a temperature of 20° C.; the mixing is performed using a mechanical stirrer equipped with a propeller-type stirring spindle at a speed of 100 rpm. The glycerol and water are mixed at ambient temperature in a beaker using a mechanical stirrer at a speed of 2000 rpm and the thickening agent is then added gradually. The stirring is maintained for a duration which makes it possible to obtain an aqueous phase which is in the form of a homogeneous gel. The fatty phase is added in one go to the aqueous gel at ambient temperature and at a moderate stirring speed (75 to 300 rpm) using a stirrer equipped with an anchor-type spindle. This stirring is then maintained for 10 minutes and no cooling step is necessary.

TABLE 1

| | (F₁) | (F₂) | (F₃) | (F′₁) | (F′₂) | (F′₃) | (F′₄) |
|---|---|---|---|---|---|---|---|
| Fatty phase | | | | | | | |
| Lanol ™ 2681 ⁽¹⁾ | 15% | 16% | 15% | 10% | 8% | 8% | 8% |
| Sepineo ™ SE 68⁽²⁾ | 1% | 1% | 0% | 1% | 1% | 1% | 0% |
| Montanov ™L⁽⁷⁾ | 0% | 0% | 1% | 0% | 0% | 0% | 0% |
| Sepicide ™ HB ⁽³⁾ | 1% | 1% | 1% | 1% | 1% | 1% | 1% |
| Simaline ™WO⁽⁴⁾ | 3% | 3% | 3% | 3% | 3% | 3% | 3% |
| Montane ™80⁽⁶⁾ | 0% | 0% | 0% | 0% | 0% | 0% | 1% |
| Aqueous phase | | | | | | | |
| Diclofenac sodium | 1% | 1% | 1% | 0% | 0% | 1% | 1% |
| Sepineo ™P600⁽⁵⁾ | 4% | 4% | 4% | 4% | 4% | 4% | 4% |
| Water | q.s. | q.s. | q.s. | q.s. | q.s. | q.s. | q.s. |
| | 100% | 100% | 100% | 100% | 100% | 100% | 100% |

| | (F′₅) | (F′₆) | (F′₇) |
|---|---|---|---|
| Fatty phase | | | |
| Lanol ™ 2681 ⁽¹⁾ | 10% | 10% | 10% |
| Sepineo SE 68⁽²⁾ | 1% | 2% | 1% |
| Sepicide ™ HB ⁽³⁾ | 1% | 1% | 1% |
| Simaline ™WO⁽⁴⁾ | 3% | 3% | 3% |
| Montane ™80⁽⁶⁾ | 0% | 0% | 0% |
| Aqueous phase | | | |
| Diclofenac sodium | 1% | 1% | 1% |
| Sepineo ™ P600⁽⁵⁾ | 4% | 4% | 5% |
| Water | q.s. 100% | q.s. 100% | q.s. 100% |

⁽¹⁾ Lanol ™ 2681, or Coco-Caprylate/Caprate.
⁽²⁾Sepineo ™ SE 68 is a mixture comprising, per 100% of its weight, from 78% to 85% by weight of a mixture of n-hexadecanol and n-octadecanol, and from 15% to 22% by
weight of a mixture of n-hexadecyl glucoside with a mean degree of polymerization of 1.20 and n-octadecyl glucoside with a mean degree of polymerization of 1.20, used as emulsifying agent.
⁽³⁾ Sepicide ™ HB is a mixture of phenoxyethanol, methylparaben, ethylparaben, butylparaben and n-propylparaben, used as a preservative.
⁽⁴⁾Simaline ™WO, or PEG 30 Dipolyhydroxystearate, is an emulsifying surfactant.
⁽⁵⁾Sepineo ™P600 is a self-invertible inverse latex comprising, per 100% of its weight, between 30% and 40% by weight of a crosslinked copolymer of acrylamide and of sodium acryloyldimethyltaurate, used as a thickening agent.
⁽⁶⁾Montane ™80 is a composition comprising sorbitan monooleate, used as water-in-oil type emulsifying agent.
⁽⁷⁾Montanov ™L is a composition comprising, per 100% of its weight, from 80% by weight to 90% by weight of a mixture of dodecyl alcohol, myristyl alcohol, hexadecyl alcohol, behenyl alcohol and arachidyl alcohol, and 10% by weight to 20% by weight of a
mixture of dodecyl glucoside with a mean degree of polymerization of 1.20, dodecyl glucoside with a mean degree of polymerization of 1.20, tetradecyl glucoside with a mean degree of polymerization of 1.20, hexadecyl glucoside with a mean degree of polymerization of 1.20, arachidyl glucoside with a mean degree of polymerization of 1.20 and behenyl glucoside with a mean degree of polymerization of 1.20.

2 Demonstration of the Properties of the Water-in-Oil Emulsions (F₁) to (F₃) According to the Invention and of the Water-in-Oil Emulsions (F′₁) to (F′₇) According to the Prior Art.
2.1 Characterization of the Appearance and the Viscosity of the Water-in-Oil Emulsions (F₁) to (F₃) According to the Invention and of the Water-in-Oil Emulsions (F′₁) to (F′₇) According to the Prior Art.

The emulsions (F1) to (F3) and (F′1) to (F′7) obtained according to the process described above are then stored in an insulated climatic chamber regulated at a temperature of 25° C. for 7 days. After the conclusion of this period of 7 days, the appearance (APP) of each emulsion prepared is observed and the dynamic viscosity (p) of each emulsion is measured (in mPas). 20 The emulsions are then returned and stored in the same insulated climatic chamber regulated at a temperature of 25° C. up to 3 months. After a period of one month, each emulsion is removed from the climatic chamber in order to observe the appearance of each emulsion and in order to measure the dynamic viscosity thereof at 25° C., using a viscometer at 25° C. (Brookfield LVT, speed 6).

An aliquot of these same emulsions (F₁) to (F₃) and (F′₁) to (F′₇) obtained according to the process described above are also stored in an insulated climatic chamber regulated at a temperature of 45° C. for 7 days. After the conclusion of this period of 7 days, the appearance (APP) of each emulsion prepared is observed and the dynamic viscosity (p) of each emulsion is measured (in mPas).

2.2 Characterization of the Direction of the Emulsions (F₁) to (F₃) According to the Invention and of the Emulsions (F′₁) to (F′₇) According to the Prior Art.

The conductivity (σ) of the emulsions (F₁) to (F₃) according to the invention and of the emulsions (F′₁) to (F′₇) is measured at 25° C., after a period of storage of said emulsions of one day in an insulated climatic chamber regulated at a temperature of 25° C., by means of an LF 196™ brand conductivity meter from WTW equipped with a TetraCon™ 96 electrode.

If, for a given emulsion, (σ)≤0.5 μS·cm⁻¹, the emulsion is considered to be non-conductive and consequently the external phase is considered not to be the aqueous phase but the oily phase.

If, for a given emulsion, (σ)>0.5 μS·cm⁻¹, the emulsion is considered to be conductive and consequently the external phase is considered not to be the oily phase but the aqueous phase.

This same measurement of the conductivity of the emulsions (F₁) to (F₃) according to the invention and of the emulsions (F′₁) to (F′₇) is measured at 25° C. after 7 days and 3 months at 25° C., and after 7 days at 45° C.

2.3 Results Obtained for the Water-in-Oil Emulsions (F₁) to (F₃) According to the Invention and for the Water-in-Oil Emulsions (F′₁) to (F′₇) According to the Prior Art.

The evaluation methods described in paragraphs 2.1 and 2.2 were applied to the water-in-oil emulsions (F₁) to (F₃) according to the invention and to the water-in-oil emulsions (F′₁) to (F′₇) according to the prior art. The results obtained are recorded in table 2 below.

TABLE 2

| | (F₁) | | (F₂) | | (F₃) | |
|---|---|---|---|---|---|---|
| (APP) at 1 day | Homogeneous | | Homogeneous | | Homogeneous | |
| (visual) | liquid | | liquid | | liquid | |
| (σ) at 1 day | ≤0.5 | μS · cm⁻¹ | ≤0.5 | μS · cm⁻¹ | ≤0.5 | μS · cm⁻¹ |
| at 25° C. | | | | | | |
| (APP) at 7 days | Homogeneous | | Homogeneous | | Homogeneous | |
| at 25° C. | liquid | | liquid | | liquid | |
| (σ) at 7 days | ≤0.5 | μS · cm⁻¹ | ≤0.5 | μS · cm⁻¹ | ≤0.5 | μS · cm⁻¹ |
| at 25° C. | | | | | | |
| (μ) at 7 days | 40 000 | mPas | 30 500 | mPas | 30 000 | mPas |
| at 25° C. | | | | | | |
| (APP) at 7 days | Homogeneous | | Homogeneous | | Homogeneous | |
| at 45° C. | liquid | | liquid | | liquid | |
| (σ) at 7 days | ≤0.5 | μS · cm⁻¹ | ≤0.5 | μS · cm⁻¹ | ≤0.5 | μS · cm⁻¹ |
| at 45° C. | | | | | | |
| (μ) at 7 days | 14 000 | mPas | 13 000 | mPas | 13 000 | mPas |
| at 45° C. | | | | | | |
| (APP) at 3 | Homogeneous | | Homogeneous | | Homogeneous | |
| months | liquid | | liquid | | liquid | |
| (σ) at 3 months | ≤0.5 | μS · cm⁻¹ | ≤0.5 | μS · cm⁻¹ | ≤0.5 | μS · cm⁻¹ |
| at 25° C. | | | | | | |
| (μ) at 3 | 35 000 | mPas | 16 400 | mPas | 14 000 | mPas |
| months | | | | | | |

| | (F′₁) | | (F′₂) | | (F′₃) | |
|---|---|---|---|---|---|---|
| (APP) at 1 day | Homogeneous | | Homogeneous | | Homogeneous | |
| (visual) | liquid | | liquid | | liquid | |
| (σ) at 1 day | ≤0.5 | μS · cm⁻¹ | ≤0.5 | μS · cm⁻¹ | >0.5 | μS · cm |
| at 25° C. | | | | | | |
| (APP) at 7 days | Homogeneous | | Homogeneous | | Homogeneous | |
| at 25° C. | liquid | | liquid | | liquid | |
| (σ) at 7 days | ≤0.5 | μS · cm⁻¹ | ≤0.5 | μS · cm⁻¹ | >0.5 | μS · cm⁻¹ |
| at 25° C. | | | | | | |
| (μ) at 7 days | 100 000 | mPas | 130 000 | mPas | 225 000 | mPas |
| at 25° C. | | | | | | |
| (APP) at 7 days | Homogeneous | | Homogeneous | | Homogeneous | |
| at 45° C. | liquid | | liquid | | liquid | |
| (σ) at 7 days | ≤0.5 | μS · cm⁻¹ | ≤0.5 | μS · cm⁻¹ | >0.5 | μS · cm⁻¹ |
| at 45° C. | | | | | | |
| (μ) at 7 days | 62 000 | mPas | 85 000 | mPas | 100 000 | mPas |
| at 45° C. | | | | | | |
| (APP) at 3 | Homogeneous | | Homogeneous | | Homogeneous | |
| months | liquid | | liquid | | liquid | |
| (σ) at 3 months | ≤0.5 | μS · cm⁻¹ | ≤0.5 | μS · cm⁻¹ | >0.5 | μS · cm⁻¹ |
| at 25° C. | | | | | | |

| | | | | | |
|---|---|---|---|---|---|
| (μ) at 3 | 120 000 | mPas | 110 000 | mPas | not measured |
| months | | | | | due to phase |
| | | | | | inversion |

| | (F′₄) | (F′₅) | (F′₆) | (F′₇) |
|---|---|---|---|---|
| (APP) at 1 day | Homogeneous | Homogeneous | Homogeneous | Homogeneous |
| (visual) | liquid | liquid | liquid | liquid |
| (σ) at 1 day at | >0.5 μS · cm⁻¹ | ≤0.5 μS · cm⁻¹ | ≤0.5 μS · cm⁻¹ | ≤0.5 μS · cm⁻¹ |
| 25° C. | | | | |
| (APP) at 7 days at | Homogeneous | Homogeneous | Homogeneous | Homogeneous |
| 25° C. | liquid | liquid | liquid | liquid |
| (σ) at 7 days at | >0.5 μS · cm⁻¹ | ≤0.5 μS · cm⁻¹ | ≤0.5 μS · cm⁻¹ | ≤0.5 μS · cm⁻¹ |
| 25° C. | | | | |
| (μ) at 7 days at | not measured | 140 000 mPas | 100 000 mPas | 140 000 mPas |
| 25° C. | as an O/W | | | |
| | emulsion was | | | |
| | obtained | | | |
| (APP) at 7 days | Homogeneous | Homogeneous | Homogeneous | Homogeneous |
| at 45° C. | liquid | liquid | liquid | liquid |
| (σ) at 7 days at | >0.5 μS · cm⁻¹ | >0.5 μS · cm⁻¹ | >0.5 μS · cm⁻¹ | >0.5 μS · cm⁻¹ |
| 45° C. | | | | |
| (μ) at 7 days at | not measured | not measured | 110 000 mPas | 135 000 mPas |
| 45° C. | as an O/W | due to phase | | |
| | emulsion was | inversion | | |
| | obtained | | | |
| (APP) at 3 months | Homogeneous | Homogeneous | Homogeneous | Homogeneous |
| | liquid | liquid | liquid | liquid |
| (σ) at 3 months at | >0.5 μS · cm⁻¹ | >0.5 μS · cm⁻¹ | >0.5 μS · cm⁻¹ | >0.5 μS · cm⁻¹ |
| 25° C. | | | | |
| (μ) at 3 months | not measured | not measured | not measured | not measured |
| | as an O/W | due to phase | due to phase | due to phase |
| | emulsion was | inversion | inversion | inversion |
| | obtained | | | |

### 2.4 Analysis of the Results

The water-in-oil emulsions (F₁), (F₂) and (F₃) according to the invention are therefore characterized:
by a stability of their water-in-oil form after 3 months of storage at a temperature of 25′C; the observed appearance after this period of storage is still homogeneous;
by a stability of their water-in-oil form after 7 days of storage at a temperature of 45° C.; the observed appearance after this period of storage is still homogeneous;
by dynamic viscosity values measured after 7 days of storage at 25° C. by means of a Brookfield LV viscometer at 25° C. and at a speed of 6 rpm of between 30 000 mPa·s (for (F₃)) and 40 000 mPa·s (for (F₁)).

The emulsions (F′₁) and (F′₂) differ from the emulsions according to the invention by the absence of diclofenac sodium, but stability of their water-in-oil form after 3 months of storage at a temperature of 25° C. and after 7 days of storage at a temperature of 45° C.

Formulation (F′₅) differs from formulation (F′₁) only by the presence of 1% diclofenac.

Formulation (F′₃) differs from formulation (F′₂) only by the presence of 1% diclofenac.

However, (F′₅) and (F′₃) are not stable after a period of storage of 7 days at 45° C., taking an oil-in-water form and no longer a water-in-oil form. This observation demonstrates the instability caused by the presence of the diclofenac sodium, and the stability problem is resolved under the conditions of formulations (F₁), (F₂), and (F₃).

1. A pharmaceutical composition suitable for topical use which is in the form of an emulsion of water-in-oil type comprising, per 100% of weight:
from 60% to 80% by weight of a gelled aqueous phase (A1) comprising a nonsteroidal inflammatory substance,
from 20% to 40% by weight of a fatty phase (A2) comprising at least one oil and an emulsifying system comprising a combination of at least one emulsifying surfactant (S₁) selected from the elements of the group consisting of compositions of alkyl polyglycosides, and compositions of alkyl polyglycosides and of fatty alcohols, and of at least one emulsifying surfactant (S₂) selected from the elements of the group consisting of polyglycerol esters, alkoxylated polyglycerol esters, polyglycol polyhydroxystearates, polyglycerol polyhydroxystearates, and alkoxylated polyglycerol polyhydroxystearates.

2. The pharmaceutical composition as claimed in claim 1, wherein the gelled aqueous phase comprises, per 100% of its weight:
from 0.625% to 10% by weight of a crosslinked anionic polyelectrolyte,
from 0.625% to 5% by weight of the anti-inflammatory substance,
from 85% to 98.75% by weight of water.

3. The pharmaceutical composition as claimed in claim 2, wherein the crosslinked anionic polyelectrolyte comprises a proportion of greater than or equal to 25 mol % of monomer units derived from 2-methyl-2-[(1-oxo-2-propenyl)amino]-1-propanesulfonic acid in free acid or partially or totally salified form.

4. The pharmaceutical composition as claimed in claim 1, wherein the fatty phase (A2) comprises from 1.25% to 25% by weight of the emulsifying system and from 75% to 98.75% by weight of at least one oil.

5. The pharmaceutical composition as claimed in claim 1, wherein the emulsifying system comprises, per 100% by weight, from 12% to 88% by weight of surfactant (S1) and from 12% to 88% by weight of surfactant (S2).

6. The pharmaceutical composition as claimed in claim 1, wherein the anti-inflammatory substance is chosen from the alkali metal, alkaline earth metal, ammonium, N,N-dialkylammonium and N,N,N-trialkylammonium salts, the alkyl groups of these each comprising between 1 and 4 carbon atoms, of the elements of the group consisting of 2-[2-(2,6-dichlorophenyl)aminophenyl]ethanoic acid (CAS number=15307-86-5) or diclofenac of formula (Ia): 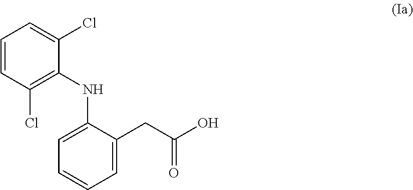 [SMILES: O=C(O)Cc1ccccc1Nc1c(Cl)cccc1Cl]
2-[2-[2-(2,6-dichloroanilino)phenyl]acetyl]oxyacetic acid (CAS number=89796-99-6) or aceclofenac of formula (Ib), 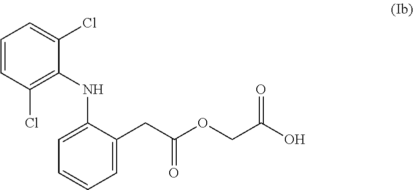 [SMILES: O=C(O)COC(=O)Cc1ccccc1Nc1c(Cl)cccc1Cl]
2-(5-benzoylthiophen-2-yl)propanoic acid (CAS number=33005-95-7 (RS)) or tiaprofenic acid in the R enantiomer form of formula (Ic1) and in the S enantiomer form of formula (Ic2) 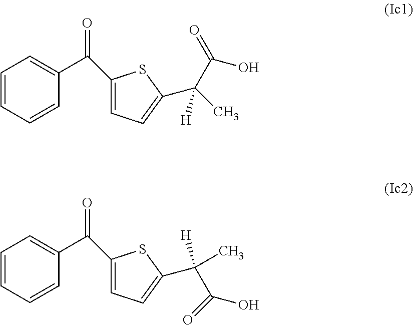 [SMILES: C[C@H](C(=O)O)c1ccc(C(=O)c2ccccc2)s1 ; C[C@@H](C(=O)O)c1ccc(C(=O)c2ccccc2)s1]
2-[4-(2-methylprop-2-enylamino)phenyl]propanoic acid (CAS number=39718-89-3) or alminoprofen of formula (Id), 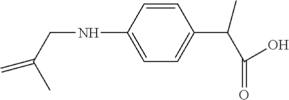 [SMILES: C=C(C)CNc1ccc(C(C)C(=O)O)cc1]
2-(1,8-diethyl-4,9-dihydro-3H-pyrano[3,4-b]indol-1-yl)acetic acid (CAS number=41340-25-4) or etodolac of formula (Ie), 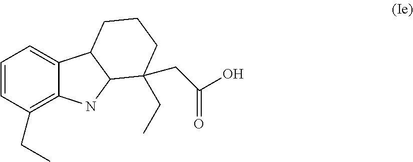 [SMILES: CCc1cccc2c1NC1C2CCCC1(CC)CC(=O)O]
(±)-2-fluoro-α-methyl-(1,1′-biphenyl)-4-acetic acid or flurbiprofen in the R enantiomer form of formula (If1) and in the S enantiomer form of formula (If2), (CAS number=5104-49-4 (RS)): 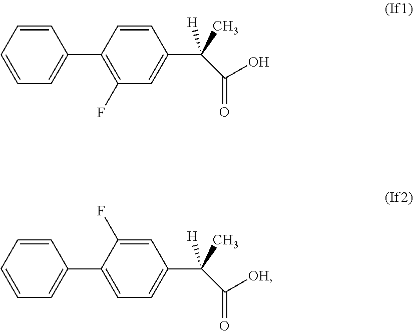 [SMILES: C[C@@H](C(=O)O)c1ccc(-c2ccccc2)c(F)c1 ; C[C@@H](C(=O)O)c1ccc(-c2ccccc2)c(F)c1]
2-[4-(2-methylpropyl)phenyl]propanoic acid or ibuprofen in the R enantiomer form of formula (Ig1) and in the S enantiomer form of formula (Ig2), (CAS number=15687-27-1 (RS)): 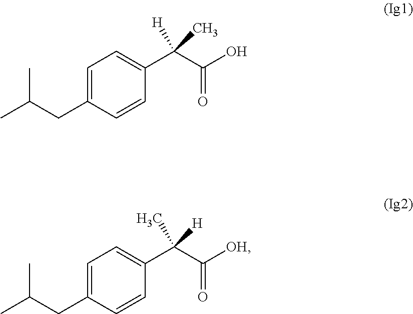
2-(3-benzoylphenyl)propionic acid in the S(+) enantiomer form (CAS number=22161-81-5) and R(−) enantiomer form (CAS number=56105-81-8) and in the form of a racemic mixture (CAS number=22071-15-4) or ketoprofen of formula (Ih), 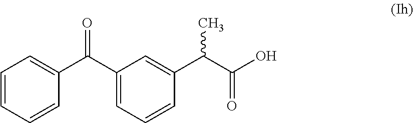 [SMILES: CC(C(=O)O)c1cccc(C(=O)c2ccccc2)c1]
6-methoxy-α-methyl-2-naphthaleneacetic acid or naproxen in the S(+) enantiomer form (CAS number=22204-53-1) of formula (Ii) or in the form of a racemic mixture (CAS number=23981-80-8):

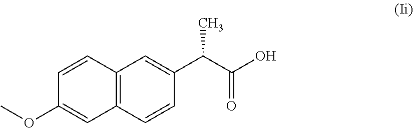 [SMILES: COc1ccc2cc([C@H](C)C(=O)O)ccc2c1]

7. The pharmaceutical composition as claimed in claim 1, wherein the anti-inflammatory substance is chosen from the sodium salt of 2-[2-(2,6-dichlorophenyl)aminophenyl]ethanoic acid, the diethylammonium salt of 2-[2-(2,6-dichlorophenyl)aminophenyl]ethanoic acid, the sodium salt of 2-[2-[2-(2,6-dichloroanilino)phenyl]acetyl]oxyacetic acid, the diethylammonium salt of 2-[2-[2-(2,6-dichloroanilino)phenyl]acetyl]oxyacetic acid, the sodium salt of 2-[4-(2-methylpropyl)phenyl]propanoic acid, and the sodium salt of 2-(3-benzoylphenyl)propionic acid.

8. The pharmaceutical composition as claimed in claim 1, wherein the emulsifying surfactant (S1) consists of at least one alkyl polyglycoside composition (C1) represented by the formula (VIII):
R1-O-(G)x-H (VIII)
in which x represents a decimal number between 1.05 and 2.5, G represents the glucosyl or β-D-glucopyranosyl radical, obtained from the removal of the hemiacetal hydroxyl group from β-D-glucopyranose, and R1 represents a radical chosen from the elements of the group consisting of the radicals n-dodecyl, n-tetradecyl, n-hexadecyl, n-octadecyl, n-eicosyl and n-behenyl, said composition (C1) consisting of a mixture of compounds represented by the formulae (VIII1), (VIII2), (VIII3), (VIII4) and (VIII5):
R1-O-(G)1-H (VIII1)
R1-O-(G)2-H (VIII2)
R1-O-(G)3-H (VIII3)
R1-O-(G)4-H (VIII4)
R1-O-(G)5-H (VIII5)
in the respective molar proportions a1, a2, a3, a4 and a5, such that:
the sum a1+a2+a3+a4+a5 is equal to 1, and that
the sum a1+2a2+3a3+4a4+5a5 is equal to x.

9. The pharmaceutical composition as claimed in claim 1, wherein the emulsifying surfactant (S1) consists of at least one composition (C2) comprising, per 100% of weight:
from 10% to 50% by weight of at least one alkyl polyglycoside composition (C1) represented by formula (VIII):
R1-O-(G)x-H (VIII)
in which x represents a decimal number between 1.05 and 2.5, G represents a glucose residue, and R1 represents a radical chosen from the elements of the group consisting of the radicals n-dodecyl, n-tetradecyl, n-hexadecyl, n-octadecyl, n-eicosyl and n-behenyl, said composition consisting of a mixture of compounds represented by the formulae (VIII1), (VIII2), (VIII3), (VIII4) and (VIII5):
R1-O-(G)1-H (VIII1)
R1-O-(G)2-H (VIII2)
R1-O-(G)3-H (VIII3)
R1-O-(G)4-H (VIII4)
R1-O-(G)5-H (VIII5)
in the respective molar proportions a1, a2, a3, a4 and a5, such that:
the sum a1+a2+a3+a4+a5 is equal to 1, and that
the sum a1+2a2+3a3+4a4+5a5 is equal to x; and
from 90% to 50% by weight of at least one fatty alcohol of formula (IX):
R′1-OH (IX),
in which R′1 represents a radical chosen from the elements of the group consisting of the radicals n-dodecyl, n-tetradecyl, n-hexadecyl, n-octadecyl, n-eicosyl and n-behenyl, where R′1 may be identical to or different from R1.

10. The pharmaceutical composition as claimed in claim 1, wherein the emulsifying surfactant (S₂) consists of at least one polyglycol polyhydroxystearate represented by the formula (XII): 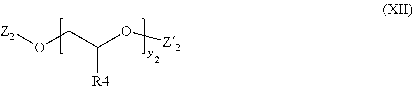
in which y₂ represents an integer greater than or equal to 2 and less than or equal to 50, R₄ represents a hydrogen atom, a methyl radical or an ethyl radical, and Z₂ represents a radical of formula (XIII): 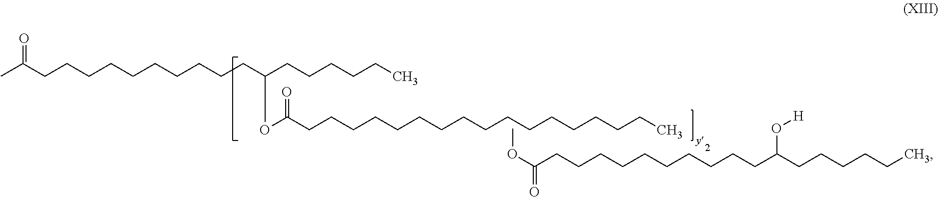
in which y′₂ represents an integer greater than or equal to 0 and less than or equal to 10, and Z′₂ represents a radical of formula (XIII) as defined above, where Z₂′ may be identical to or different from Z₂, or a hydrogen atom.

11. The pharmaceutical composition as claimed in claim 1, wherein the weight ratio between the emulsifying surfactant (S1) and the emulsifying surfactant (S2) is greater than or equal to ¼ and less than or equal to 1.

12. The pharmaceutical composition as claimed in claim 1, wherein said composition is intended to be used for therapy in a human being or animal.

13. A method for reducing and/or eliminating local pains, post-traumatic inflammation of the joints, muscles, tendons or ligaments, localized forms of soft-tissue rheumatism, localized forms of osteoarthritis, actinic keratosis caused by overexposure to sunlight, acute migraine, pain associated with bone metastases, fever due to malignant lymphogranulomatosis (Hodgkin's lymphoma), multi-drug resistant E. coli, Shy-Drager syndrome and diabetes mellitus, comprising topically applying an effective amount of the pharmaceutical composition of claim 1 to a patient in need thereof.

14. The pharmaceutical composition as claimed in claim 1, further comprising at least one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizing agents, opacifying agents, pearlescent agents, superfatting agents, sequestering agents, chelating agents, antioxidants, fragrances, essential oils, preservatives, conditioning agents, deodorants, mineral fillers or pigments.

15. A device which is in a form chosen from a pot, a pump-bottle, a wipe, a mask, a transdermal device, a patch, a poultice, a compress, a tube, spray, said device comprising the pharmaceutical composition as defined in claim 1.

16. The pharmaceutical composition as claimed in claim 1, wherein the fatty phase (A2) comprises from 1.25% to 25% by weight of the emulsifying system and from 75% to 98.75% by weight of at least one oil and of at least one wax.

17. The pharmaceutical composition as claimed in claim 1, wherein the fatty phase (A2) comprises from 1.25% to 20% by weight of the emulsifying system and from 80% to 98.75% by weight of at least one oil.

18. The pharmaceutical composition as claimed in claim 1, wherein the fatty phase (A2) comprises from 1.25% to 20% by weight of the emulsifying system and from 82% to 98.75% by weight of at least one oil.

19. The pharmaceutical composition as claimed in claim 2, wherein the emulsifying system comprises, per 100% by weight, from 12% to 88% by weight of surfactant (S1) and from 12% to 88% by weight of surfactant (S2).

20. The pharmaceutical composition as claimed in claim 3, wherein the emulsifying system comprises, per 100% by weight, from 12% to 88% by weight of surfactant (S1) and from 12% to 88% by weight of surfactant (S2).
